(19) United States
(12) Patent Application Publication
(10) Pub. No.: US 2015/0160242 A1
(43) Pub. Date: Jun. 11, 2015

(54) POLYMER MATRIX WITH TARGET BINDING SITE FOR CREATININE AND DERIVATIVES THEREOF, ITS PREPARATION AND USES

(71) Applicant: FRAUNHOFER-GESELLSCHAFT ZUR FÖRDERUNG DER ANGEWANDTEN FORSCHUNG E.V., München (DE)

(72) Inventors: Umporn Athikomrattanakul, Potsdam-Golm (DE); Nenad Gajovic-Eichelmann, Berlin (DE)

(21) Appl. No.: 14/400,926

(22) PCT Filed: Nov. 16, 2012
(86) PCT No.: PCT/EP2012/072903

§ 371 Date: Nov. 13, 2014

(30) Foreign Application Priority Data
May 16, 2012 (EP) 12168400.5

(51) Int. Cl. G01N 33/70; C07D 233/88; C08F 222/10

(52) U.S. Cl. CPC G01N 33/70; C08F 222/1006; C07D 233/88; C08F 2222/104

(57) ABSTRACT

This invention provides a polymer matrix with target binding site and methods for preparing the same. This invention further provides uses of the polymer matrix in the separation or extraction of creatinine from complex mixtures, and in methods, kits and devices for detecting creatinine content in samples.

### BACKGROUND

Creatinine levels in biological fluids are a very important parameter for clinical routine diagnostics. The normal concentration range of creatinine in the serum of healthy adults is 62-124 μM (˜0.5-1.5 mg/dL) whereas in urine it is in the range of 25-400 mg/dL (one-off collection) or 500-2000 mg over 24 hours (Walsh, D. A., Dempsey, E., Anal. Chim. Acta, 459:187-198 (2002)). The current method for creatinine determination in clinical laboratories is a spectrophotometric technique based on the colorimetric Jaffe reaction of creatinine with alkaline picrate. Before measurement, sample pretreatment with some steps to remove interferences in serum or plasma is necessary, for example, centrifugation, extraction and/or filtration. However, the interferences from endogeneous species can still corrupt the creatinine analysis by Jaffe method because of its general lack of specificity and selectivity.

Creatinine detection in biological fluids is one of the most important clinical diagnostic parameters. The creatinine level is an index for the determination of renal, thyroid and muscular functions and even myocardial infarction. In the case of kidney failure or muscle disorder, elevated levels of creatinine in blood or urine can be determined. A method based on Jaffe's reaction has been proposed since 1886 (Jaffe, M. Z. Z. Physiol. Chem., 10: 391-400 (1886)) and has since become a commercially available analysis that is routinely used in clinical laboratories today.

The reaction between creatinine and picric acid in alkaline solution leads to an orange-red complex formation, which can be detected using a spectrophotometer. However, the main drawback of this method is the low selectivity and specificity. Many interfering factors can easily impair the measurement. The interferences originate not only from other metabolites such as bilirubin, creatine and uric acid, but also from other rather unrelated biological compounds such as glucose, protein or acetoacetate, which can all lead to false results (Weber, J. A., van Zanten, A. P., Clin. Chem., 37: 695-700 (1991); Bishop, M. L., et al., Clinical Chemistry, Principles, Procedures, Correlations, 4th ed., Lippincott Williams and Wilkins, New York, 2000, p. 440 (Chapter 21)).

Some additional developments for the Jaffe reaction were reported. Jiugao et al. used cellulose nitrate to adsorb creatinine (Jiugao, Y., et al., Carbohyd. Polym., 70: 8-14 (2007)), but the method suffered from low adsorption amounts of creatinine, Grabowska et al. applied a flow-through microsystem (Grabowska, I., et al., Anal. Chim. Acta, 540: 181-185 (2005)), which was interfered by glucose at 1 g/L in the sample, and the analyte flow rate was very low compared to flow injection analysis. Fossati and colleagues proposed a method using three enzymes such as creatininase, creatinase and sarcosine oxidase or creatinine deiminase to catalyse the hydrolysis of creatinine. The ammonia or hydrogen peroxide product was detected by an analyzer, spectrophotometer or electrochemical methods (Fossati, P., et al., Clin. Chem. 29: 1494-1496 (1993); Kubo, I., Anal. Chim. Acta, 187: 31-37 (1986); U.S. Pat. No. 5,958,786; US 2003/0070548A1; US 2009/0045056A1; Yadav, S., Kumar, A., Pundir, C. S., Anal. Biochem., 419: 277-283 (2011)). The drawback of such enzymatic methods are that they are time consuming and costly, and moreover suffer from the poor stability of the enzymes as well as interference by disintegration of ammonia in the sample.

Creatinine from complex solutions has been measured by HPLC but this method needs sample pretreatment and requires skilled personnel and considerable technical kit.

Furthermore, a highly specific antibody has been produced against creatinine and applied in an amperometric sensor or surface plasmon resonance (Benkert, A. B., et al., Anal. Chem., 72: 916-921 (2000); Stocklein, W. F. M., et al., Biosens. Bioelctron., 15: 377-382 (2000)). The antibody-based method, however, was never commercialized because antibody production is expensive whilst at the same time the antibody-based detection of high-abundance analytes like creatinine requires a large amount of antibody per analysis.

Furthermore, molecularly imprinted polymers (MIPs) as artificial receptors for creatinine have been described. MIPs can have antibody-like properties, in that they can provide selective or even specific binding sites for the target molecule. However, MIPs consist of a highly crosslinked polymer rather than a protein backbone. MIPs are therefore usually rigid, sturdy materials which can be used in conditions in which it would be impossible to use biomolecules like antibodies, e.g. in organic solvents or at elevated temperatures et cetera. MIPs are also commercially attractive, because they can be synthesized at a much lower price per milligram than antibodies.

The MIP-based detection of creatinine was realized using methods such as UV-Vis spectrometry and/or HPLC (Sreenivasan, K., Sivakumar, R., J. Appl. Polym. Sci., 66: 2539-2542 (1997); Subat, M, Borovik, A. S., Konig, B., J. Am. Chem. Soc. 126: 3185-3190 (2004); Tsai, H. A., Syu, M. J., Biomaterials, 26: 2759-2766 (2005); Tsai, H. A., Syu, M. J., Anal. Chim. Acta, 539: 107-116 (2005); Hsieh, R. Y., Tsai, H. A., Syu, M. J., Biomaterials, 27: 2083-2089 (2006); Lee, M. H., et al., Desalination, 234: 126-133 (2008); Chang, Y. S., et al., Anal. Chem., 81: 2098-2105 (2009); Gao, B., Li, Y., Zhang, Z., J. Chromatogr. B, 878: 2077-2086 (2010); Lee, M. H., et al., ACS Appl. Mater. Inter., 2: 1729-1736 (2010); Tsai, H. A., Syu, M. J., Chem. Eng. J., 168: 1369-1376 (2011); Li, T. J., et al., Anal. Chim. Acta, 711: 83-90 (2012)), fluorescence sensing (Subrahmanym, S., et al., Biosens. Bioelectron., 16: 631-637 (2001); Lin, H. Y., Ho, M. S., Lee, M. H., Biosens. Bioelctron., 25: 579-586 (2009); Syu, M. J., Hsu, T. J., Lin, Z. K., Anal. Chem., 82: 8821-8829 (2010)), electrochemical sensors (Panasyuk-Delaney, T., Mirsky, V. M., Wolfbeis, O. S., Electroanalysis, 14: 221-224 (2002); Lakshmi, D., Prasad, B. B., Sharma, P. S., Talanta, 70: 272-280 (2006); Sharma, P. S., Lakshmi, D., Prasad, B. B., Chromatographia, 65: 419-427 (2007); Patel, A. K., Sharma, P. S., Prasad, B. B., Electroanalysis, 20: 2102-2112 (2008); Huang, C. Y., et al., Biosens. Bioelectron., 24: 2611-2617 (2009); Khadro, B., et al., Procedia Engineering, 5:371-374 (2010); Reddy, K. K., Gobi, K. V., IPCBEE, 25: 25-29 (2011)) and ion-selective field-effect transistor (Soldatkin, A. P., et al., Talanta, 58: 351-357 (2002); Tsai, H. H., et al., Sensor. Actuat. B., 144: 407-412 (2010)).

Despite the large number of papers on MIPs able to bind creatinine to some extent and under certain conditions, none of these materials was able to solve the analytical task of sensitive and selective detection of creatinine in untreated clinical samples (plasma and/or urine) in the presence of high amounts of creatine. Other shortcomings of the previous art in molecularly imprinted polymers towards creatinine have been a low selectivity, e.g. interference from creatine and other substances, low stability, poor reproducibility, time-consuming protocols and a general incompatibility with aqueous samples, i.e. most MIPs work only in organic solvents. The latter point is a knockout-criterion for most diagnostic applications and point-of-care applications.

Description of the Invention

Until today, none of the above analytical methods has proven suitable enough for creatinine detection in order to replace the standard Jaffe reaction, which is utilised in clinical routine test. Therefore, it would be desirable to develop a simple, rapid, inexpensive, reliable method to replace or to improve the analytical quality, especially the selectivity of the Jaffe reaction.

In particular, it would be of great technical and commercial value to design and synthesize materials, which can be used to reduce the steps of sample preparation, extraction or purification. An even higher value would be added if new materials and/or methods could be combined with the Jaffe method in a way that the selectivity is significantly improved.

According to a first aspect, the invention provides a polymer matrix comprising at least one target binding site, wherein
the target binding site comprises a functional group represented by the general formula (I)

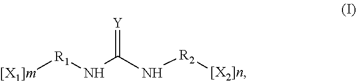

wherein
Y represents S, O or NH,
R1 and R2 are each independently selected from a group comprising aryl, heteroaryl, cycloalkyl, cycloalkenyl, alkyl, which can each be substituted and/or part of a condensed ring system. For instance, substitutions can be substitutions of H with for example C1-C6 alkyl, optionally substituted with halogen, in particular F. Substitutions can also for instance be substitutions of H with halogen, in particular F. or NO2. Substitutions can also for instance be substitutions of H with NO2. X1 and X2 each independently represent a group comprising a chemical linkage to the polymer matrix, m and n independently represent 0, 1, 2 or 3 and m+n is 1, 2, 3, 4, 5 or 6. X1 and/or X2 can therefore represent several substituents on the aryl or cycloalkylrings of R1 and/or R2.
The target binding site is suitable to bind to a target molecule represented by one of the following general formula (IIa) or (IIb), which can represent tautomeric forms of a target molecule:

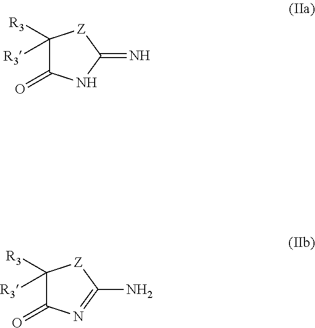

wherein Z is selected from CR4R4′, NR4″, O or S, R3, R3′, R4 and R4′ are each independently selected from a group comprising hydrogen, aryl, heteroaryl, cycloalkyl, cycloalkenyl, linear or branched alkyl, which can each be substituted and/or part of a condensed ring system,
R4″ is selected from a group comprising nitrogen protecting group, hydrogen, aryl, heteroaryl, cycloalkyl, cycloalkenyl, linear or branched alkyl, which can each be substituted and/or part of a condensed ring system.

According to a second aspect, the invention provides a polymer matrix comprising at least one target binding site, wherein
the polymer matrix is obtainable by polymerization of at least a functional monomer of the general formula (Ia)

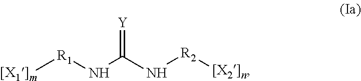

wherein
Y represents S, O or NH,
R1 and R2 are each independently selected from a group comprising aryl, heteroaryl, cycloalkyl, cycloalkenyl, alkyl, which can each be substituted and/or part of a condensed ring system, For instance, substitutions can be substitutions of H with for example C1-C6 alkyl, optionally substituted with halogen, in particular F. Substitutions can also for instance be substitutions of H with halogen, in particular F. or NO2. Substitutions can also for instance be substitutions of H with NO2. X1′ and X2′ each independently represent a polymerizable group,
m and n independently represent 0, 1, 2 or 3 and m+n is 1, 2, 3, 4, 5 or 6. X1′ and/or X2′ can therefore represent several substituents on the aryl or cycloalkylrings of R1 and/or R2.
the polymerization being performed in the presence of a target molecule represented by one of the following general formula (IIa) or (IIb), which represent tautomeric forms of the target:

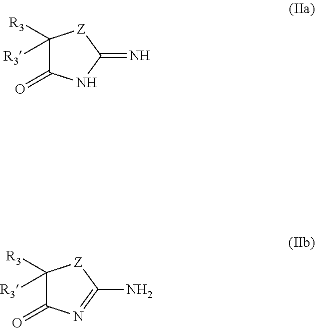

wherein Z is selected from CR4R4′, NR4″, O or S, R3, R3′, R4 and R4′ are each independently selected from a group comprising hydrogen, aryl, heteroaryl, cycloalkyl, cycloalkenyl, linear or branched alkyl, which can each be substituted and/or part of a condensed ring system,
R4″ is selected from a group comprising nitrogen protecting group, hydrogen, aryl, heteroaryl, cycloalkyl, cycloalkenyl, linear or branched alkyl, which can each be substituted and/or part of a condensed ring system,
wherein the target binding site of the polymer matrix is able to bind to the target molecule.

Binding of a target molecule means by definition of the invention the binding of the target with the polymer matrix with a measurable affinity. A measurable affinity can be for instance be characterised with the equilibrium dissociation constant KD (in mol/l) and/or by the ratio of the kinetic rate constants ka (association rate constant) and kd (dissociation rate constant). Preferably, the KD is <1 mM. More preferably, the KD is <500 ρM. Even more preferred is a KD of the polymer matrix <100 μM. Even more preferred is a KD<50 μM.

The polymerisable groups X1′ and X2′ can in principle be each moiety capable of binding which allows for fixation of the ligands in the polymer matrix. These are typically reactive moieties which can undergo a cross-linking reaction, for example with an inorganic and/or organic cross-linker. This cross-linking reaction is preferably a polymerization reaction, the term “polymerization” herein being understood as also including a polycondensation or polyaddition. For instance, both the polymerisable groups X1′ and X2′ and the cross-linker(s) bear polymerizable groups. The polymerisable groups X1′ and X2′ can for instance be chosen from the group comprising vinyl, acrylic, methacrylic, allyl or styrene groups or any other unsaturated group capable of reacting via a free-radical process, and chemical groups enabling a polycondensation, polyaddition or sol-gel reaction. Examples of suitable polymerisable groups include, e.g., acryl derivatives, methacryl derivatives such as ethylene glycol dimethacrylate or trimethylolpropane trimethacrylate, epoxides, isocyanates or allyl derivatives.

The polymerisable groups X1′ and/or X2′ can for instance further incorporate a spacer moiety, wherein the spacer moiety provides a spatial separation of the group capable of reacting via a free-radical process or the group enabling a polycondensation, polyaddition or sol-gel reaction, respectively, from the group R1 and/or R2. The spacer moiety can for instance be a saturated or unsaturated, linear or branched alkyl or alkoxy, optionally interrupted with one or more heteroatoms chosen from N, O and S. For instance, the spacer moiety can be a polyethyleneglycol.

The groups X1 and/or X2 representing groups comprising a chemical linkage to the polymer matrix can for instance be derived from the polymerisable groups X1′ and/or X2′ as defined above, after the groups X1′ and/or X2′ have been incorporated into the polymer matrix by a polymerization reaction.

Condensed ring system as used in the definitions for substituents can be for instance include naphthyl or anthracyl

Nitrogen protecting groups comprise for instance Boc, Fmoc, Acyl, carbamoyl, sulfonyl such as tosyl, benzyl, benzoyl.

In certain embodiments, the invention provides a polymer matrix, wherein the polymerization is performed in the presence of at least one cross-linker and/or initiator.

A cross-linker can for instance be an organic or an inorganic cross-linker. An inorganic cross-linker may be a bifunctional or multifunctional organosiloxane, for example.

In the case of an organic cross-linker, this may be an acryl derivative, methacryl derivative, e.g., ethylene glycol dimethacrylate or trimethylolpropane trimethacrylate, or allyl derivative, for example. To achieve a higher hydrophilicity of the polymer backbone, e.g., corresponding cross-linkers based on polyethylene glycol can be employed.

Further suitable examples of cross-linkers are apparent to those skilled in the art without any difficulty.

The polymerization reaction can be a RAFT polymerization, for example.

By means of the polymerization reaction, a polymer matrix is formed which can comprise or essentially consist of an organic or inorganic polymer, e.g., a polyurethane, polyurea, polyester, polyamide, aminoplast, epoxy resin, silicones or copolymers or mixtures thereof, in addition to the functional groups.

The cross-linking reaction, in particular, polymerization can take place as a free-radical polymerization, e.g., induced by light or thermally induced, anionically or cationically.

In certain embodiments the invention provides the polymer matrix, wherein the functional group of formula (I) is represented by the general formula (I′)

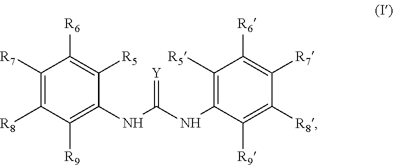

wherein R5, R6, R7, R8, R9, R5′, R6′, R7′, R8′ and R9′ each independently represent hydrogen, halogen, alkyl, halogenalkyl, NO2, CN,

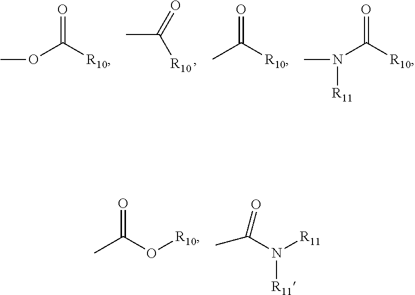

and R10, R11, R11′ being independently selected from a group comprising hydrogen, alkyl, aryl, halogenalkyl, alkylenaryl and nitrogen protecting group,
wherein at least two of R5, R6, R7, R8, R9 or at least two of R5′, R6′, R7′, R8′ and R9′can independently form part of a condensed ring system,
provided that at least one of R5, R6, R7, R8, R9 comprises X1 and/or at least one of R5′, R6′, R7′, R8′ and R9′ comprises X2.

In certain embodiments, the invention provides the polymer matrix, wherein the functional monomer of the formula (Ia) is represented by the general formula (Ia′)

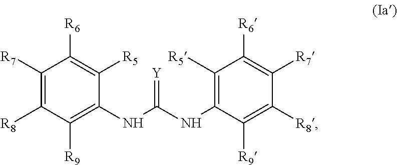

wherein R5, R6, R7, R8, R9, R5′, R6′, R7′, R8′ and R9′ each independently represent hydrogen, halogen, alkyl, halogenalkyl, NO2, CN,

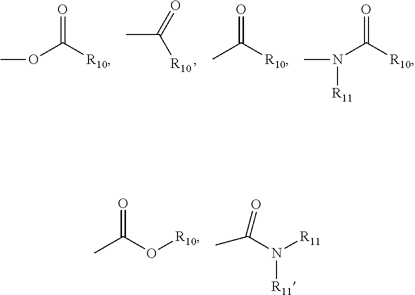

and R10, R11, R11′ being independently selected from a group comprising hydrogen, alkyl, aryl, halogenalkyl, alkylenaryl and nitrogen protecting group,
and/or wherein at least two of R5, R6, R7, R8, R9 or at least two of R5′, R6′, R7′, R8′ and R9′ independently form part of a condensed ring system,
provided that at least one of R5, R6, R7, R8, R9 comprises X1′ and/or at least one of R5′, R6′, R7′, R8′ and R9′ comprises X2′.

In certain embodiments, the invention provides the polymer matrix, wherein the functional group of formula (I′) is represented by the general formula (I″)

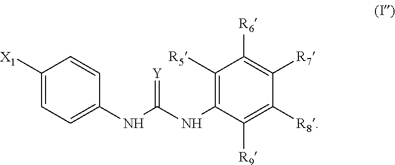

In certain embodiments, the invention provides the polymer matrix, wherein the functional monomer of formula (Ia′) is represented by the general formula (Ia″)

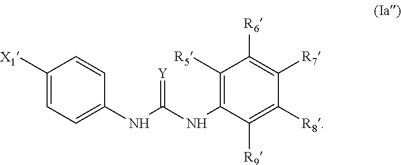

In certain embodiments, the invention provides the polymer matrix, wherein the functional group is selected from the group comprising:

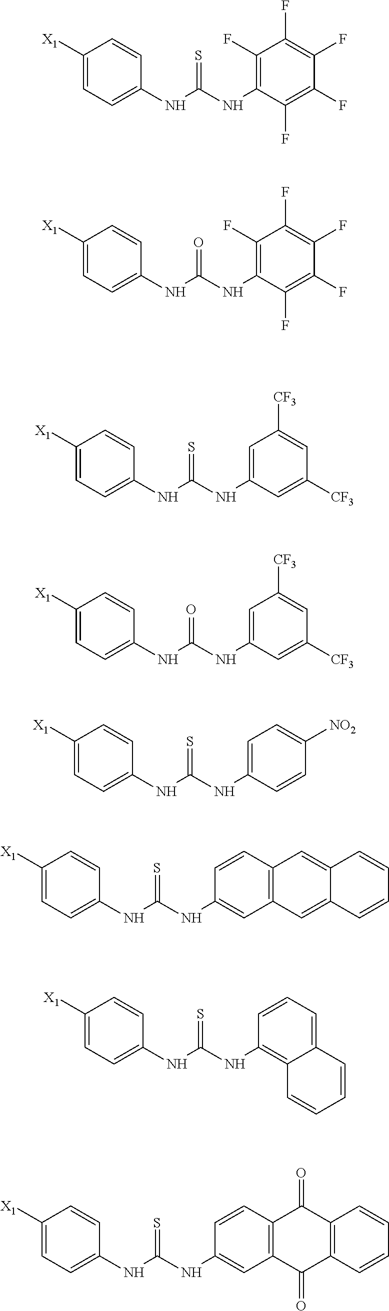

In certain embodiments, the invention provides the polymer matrix, wherein the functional monomer is selected from the group comprising:

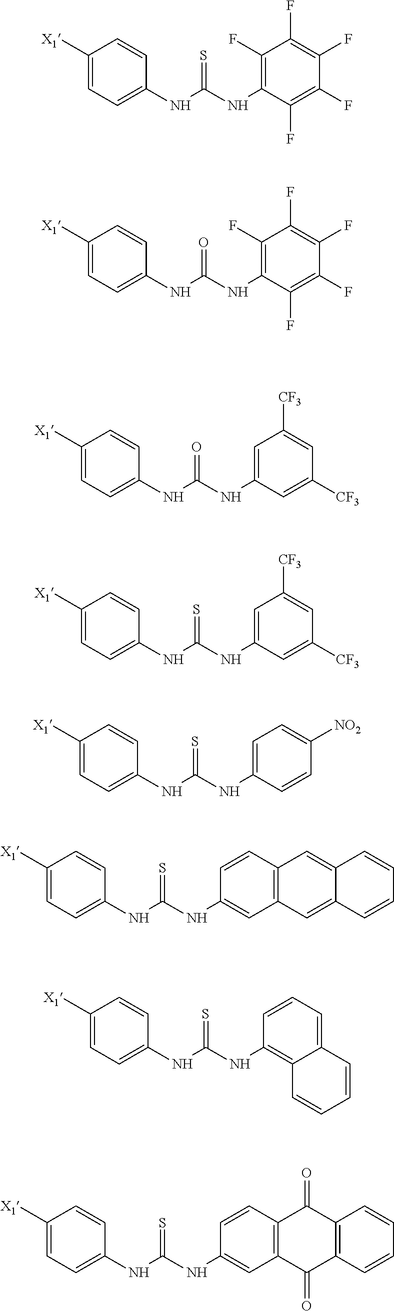

In certain embodiments, the invention provides the polymer matrix, wherein the functional group is represented by the formula (I′″)

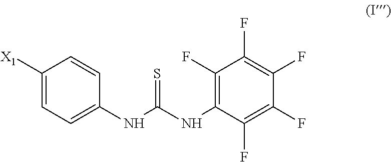

In certain embodiments, the invention provides the polymer matrix, wherein the monomer is represented by the formula (Ia′″)

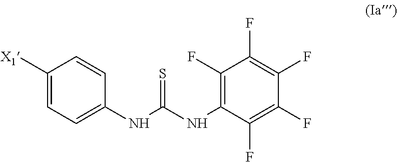

In certain embodiments of the invention, the functional group of formula (I) is represented by the formula

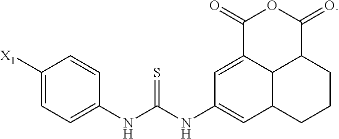

In further certain embodiments of the invention, the functional monomer of the formula (Ia) is represented by the formula

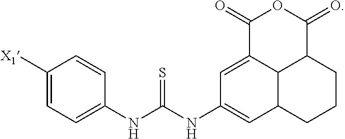

In certain embodiments, the invention provides the polymer matrix, wherein the target molecule is creatinine.

In certain embodiments, the invention provides the polymer matrix, wherein the polymer matrix is a co-polymer.

In certain embodiments, the invention provides the polymer matrix, wherein the polymerisable groups X1′ and X2′ are chosen from a group comprising unsaturated groups capable of reacting via a free-radical process, and chemical groups enabling a polycondensation, polyaddition or sol-gel reaction, in particular acryl derivatives, methacryl derivatives epoxides, isocyanates and allyl derivatives.

In certain embodiments, the invention provides the polymer matrix, wherein the X1 and X2 are derived from a polymerization reaction of a group comprising unsaturated groups capable of reacting via a free-radical process, and chemical groups enabling a polycondensation, polyaddition or sol-gel reaction, in particular acryl derivatives, methacryl derivatives epoxides, isocyanates and allyl derivatives.

According to a third aspect of the invention, the invention provides a method for the preparation of a polymer matrix as defined above, comprising the method steps of:
a) providing the at least one monomer and the at least one target molecule as defined above,
b) forming a pre-polymerization mixture by bringing said monomer and said target molecule into contact,
c) polymerizing said pre-polymerization mixture to obtain the polymer matrix.

In certain embodiments, the invention provides the method, wherein at least one cross-linker and/or initiator is added to the pre-polymerization mixture of method step b).

In certain embodiments, the invention provides the method, further comprising method step d) removing the target from the polymer matrix.

According to a fourth aspect, the invention provides a method for the separation or extraction of creatinine from a mixture comprising additional organic and/or inorganic compounds, comprising the method steps of:
a1) providing the polymer matrix as defined above,
b1) contacting the polymer matrix with the mixture and binding of creatinine with the polymer matrix,
c1) separating the polymer matrix with the bound creatinine from the mixture.

In certain embodiments, the invention provides the method, further comprising method step d1) releasing the bound creatinine from the polymer matrix.

Release of the target substance from the polymer matrix can for instance be accomplished by washing with aqueous or organic solvents, or mixtures thereof. Aqueous solvents can for instance include high ionic strength, acidic or basic solvents. Removal can be aided by temperature, e.g. by heating of the polymer matrix.

In certain embodiments, the invention provides the method, wherein the mixture is a biological specimen.

In certain embodiments, the invention provides the method, wherein the biological specimen is serum or urine.

According to a fifth aspect of the invention, the invention provides a method detection of creatinine content in a sample using the polymer matrix as defined above, the method comprising the steps of:
a2) separating a sample to be analyzed into a first and a second separate portion,
b2) using a detection method for determining a first creatinine content result in the first portion of the sample in the absence of the polymer matrix,
c2) adding the polymer matrix to the second portion of the sample,
d2) using the same detection method for determining a second creatinine content result in the second portion of the sample,
e2) substracting the second creatinine content result from the first creatinine content result to obtain the creatinine content in the sample.

Thus, provided is a method for indirect qualitative or quantitative determination of the creatinine content in a sample. The principle is based on first obtaining a gross creatinine content result from the first sample portion, i.e. a result which incorporates the detection result of the specific creatinine content and the detection result of interferences, i.e. resulting from unspecific detection of other compounds in the sample. Due to the addition of the polymer matrix to the second sample portion, creatinine is bound by the polymer matrix in a way that for instance at least part of the creatinine does not contribute to the detection result any more. This happens for instance in such a way which qualitatively or quantitatively correlates with the creatinine content in the sample portion. Therefore, second creatinine content result actually represents the detection method interferences only. The difference between the first creatinine content result and the second creatinine content result therefore represents a substraction of the detection method interference from the gross creatinine content result, wherein the difference represents the creatinine content in the sample.

In certain embodiments, the invention provides the method, wherein the detection method of method step b2) and d2) is selected from a group comprising spectrophotometric, colourimetric, fluorescence or electrochemical detection reactions.

In certain embodiments, the invention provides the method, wherein the detection method is a colourimetric detection reaction.

In certain embodiments, the invention provides the method, wherein the detection method is based on the reaction of creatinine with alkaline picrate, in particular wherein the detection method is the Jaffe reaction.

According to a further aspect of the invention, an assay kit for performing the above detection method is provided, comprising the polymer matrix as defined above and means for detecting the target molecule with the method above, wherein an assay kit is used for the detection method.

This invention also provides a method for detection of creatinine content in a sample using the polymer matrix as defined above without use of any further labelling group with the polymer matrix, the creatinine and the sample, comprising the method steps of:
a3) providing the polymer matrix,
b3) contacting the polymer matrix with the sample to be analyzed,
c3) measuring the interaction of creatinine with the polymer matrix.

As used herein, “without use of any further labelling group with the polymer matrix, the creatinine and the sample” shall mean that labelling of neither the polymer matrix, nor the creatinine, nor the sample with an additional compound or reporter group such as, for instance, a fluorescent, chemiluminescent, electrochemically active or radioactive compound or catalyst for measuring of the interaction of the creatinine with the polymer matrix is required. An additional compound or reporter group, as used in this context, shall also include an externally added reagent undergoing a measurable change in the presence or absence of creatinine, such as a colour change, absorption or emission of light, solubility change. Therefore, this aspect of the invention may also be referred to as a “label-free detection”, as opposed to a “label-based detection” which typically uses an exogenous labelling group to report the presence or absence of an analyte which is to be detected. In particular, no additional fluorescent, chemiluminescent or electrochemically active or radioactive labelling group or catalyst is present on or in either of the creatinine, the polymer matrix and the sample. Preferably, detection of creatinine content in a sample without use of any further labelling group with the polymer matrix, the creatinine and the sample shall mean measuring an inherent property of the interaction of the creatinine with the polymer matrix itself.

For example, method step c3) can comprise calorimetric measuring of heat generated by an enthalpy change due to adsorption of creatinine to the polymer matrix and/or reaction of creatinine with the polymer matrix. In particular, method step a3) can comprise providing the polymer matrix in a packed bed having a sample inlet and a sample outlet, method step b3) can comprise flowing the sample through the packed bed via the sample inlet and sample outlet, thereby contacting the polymer matrix with the sample, and method step c3) can comprise measuring the sample temperature from the sample inlet and the sample temperature from the sample outlet and deriving the creatinine content in the sample from the difference between the sample temperature at the sample inlet and the sample temperature at the sample outlet.

For example, contacting the polymer matrix with the sample in method step b3) can comprise providing the sample in a streaming medium. Preferably, the streaming medium has a fixed flow rate. The streaming medium can, for instance, comprise water, buffered solution or organic solvent, and combinations thereof.

In another embodiment of the method, method step c3) comprises measuring a spectral characteristic of the polymer matrix in contact with the sample. For example, measuring the spectral characteristic can comprise measurement of the fluorescence intensity of the polymer matrix in contact with the sample. In a preferred embodiment, the polymer matrix is intrinsically fluorescent. For instance, the fluorescence emitted by the polymer matrix decreases upon interaction with the creatinine. The fluorescence intensity can for example be measured with an excitation wavelength of 300-340 nm, preferably 310-330 nm, most preferred 315-325 nm, and an emission wavelength of 350-400 nm, preferably 360-390 nm, most preferred 365-375 nm.

The above described label-free detection of creatinine according to the present invention avoids tedious sample preparation procedures and secondary reagents and therefore renders the present method more time and cost efficient than conventional methods. Moreover, the label-free detection avoids interference which may arise from tagging creatinine or the sample with an exogenous labelling compound such as a fluorescence label, thus providing higher accuracy of the present method in comparison to label-based methods. In addition, the direct label-free detection of the present invention allows measuring the interaction of the creatinine with the polymer matrix in real-time.

Finally, this invention provides a sensing device configured for detection of creatinine content in a sample, comprising:
the polymer matrix as defined above,
means for measuring the interaction of creatinine with the polymer matrix,

wherein measuring the interaction of creatinine with the polymer matrix is without use of any further labelling group with the polymer matrix, the creatinine and the sample.

Herein, “without use of any further labelling group with the polymer matrix, the creatinine and the sample” shall be understood as described above.

In one embodiment, the means for measuring the interaction of creatinine with the polymer matrix comprises a device configured for calorimetric measuring of heat generated by an enthalpy change due to adsorption of creatinine to the polymer matrix and/or reaction of creatinine with the polymer matrix.

In another embodiment, the means for measuring the interaction of creatinine with the polymer matrix comprises a device configured for measuring of the fluorescence intensity of the polymer matrix in contact with the sample.

The generic terms polymer matrix and molecularly imprinted polymer (MIP) can be used interchangeably in the following. NIP denominates a non-imprinted polymer, i.e. a polymer matrix without produced in the absence of the target.

The present invention aims to meet the need of new method for creatinine detection or help to improve the selectivity of standard method. In order to improve the optimum binding strength and increase number of selective binding sites of molecularly imprinted polymers to the target molecule, it is important to choose the proper functional monomer (or combination thereof). Therefore, the new molecularly imprinted polymer prepared from a novel tailor-made functional monomer in this invention can provide the selective recognition for creatinine in aqueous environment (e.g. urine) better than using other commercially available functional monomers as mentioned in the literature above. In particular, this imprinted polymer in the present invention is useful as a selective extraction matrix in combination with the standard colorimetric method (Jaffe reaction) for detection and/or screening of creatinine in biological fluid. In the newly developed protocol, which we call “indirect Jaffe reaction”, two measurements are conducted and the difference between the two is providing the interference-free creatinine concentration measurement: The first measurement is the normal Jaffe reaction, which gives a signal resulting from creatinine and some interferents that were previously not separately detectable. In the second measurement, the invented creatinine MIP is added to the Jaffe reaction mixtures, and the signal derives from the interferents only, since creatinine is quantitatively bound by the MIP and no longer able to react with picric acid. The difference between the two measurements is the desired, interference-free creatinine signal. The problem from interference using Jaffe reaction can also be reduced using the imprinted polymer for a selective extraction step before the measurement. In this case, creatinine is eluted from the extraction MIP and can be quantified by the Jaffe reaction in the absence of any interferents. This method is called “direct Jaffe method with MIP-dependent solid phase extraction step”. Furthermore, this material can be prepared in other forms than the protocol presented and can be coated on any surface of the transducer for sensor application and used to measure creatinine in any solution, such as serum, plasma or urine. It could be part of products and/or systems used in the measurement in all clinical point of care monitoring of creatinine. Especially useful will be the integration of the new MIP in disposable sample collection units (e.g. urine or plasma or serum collection kits). In this way, the collection and the sample pretreatment are integrated in one device.

It is possible that this material could be developed and produced in higher amounts for industrial level for further application. It is believable that this material could be used in academic research projects, as it could be adapted to a different of measuring systems, such as optical sensing, chromatography, electrochemical sensor, etc. In addition, the advantages related to the use of MIP are high affinity and selectivity, inexpensive ingredients, simplicity of preparation, long-term storage, physical and chemical stability, which able the use thereof in various conditions and reusable several time without loss of effectiveness.

Surprisingly, the inventors have thus noted that it is possible to produce molecularly imprinted polymers (MIPs) dedicated to the selective recognition of creatinine. Therefore, according to an aspect, the present invention related to a method for preparation of imprinted polymer (MIP).

The molecularly imprinted polymers can for instance be prepared as a monolith a film or a nanoparticle.

### FIGURES

The following figures and examples are understood to be illustrative only and not intended to limit the scope of the invention.

FIG. 1 shows a creatinine binding study of the imprinted polymer and a control polymer.

FIG. 2 shows Fluorescence emission spectra obtained by titration of PTU (1 mM) with creatinine.

FIG. 3 shows an exemplary embodiment of the preparation of the polymer matrix/molecularly imprinted polymers.

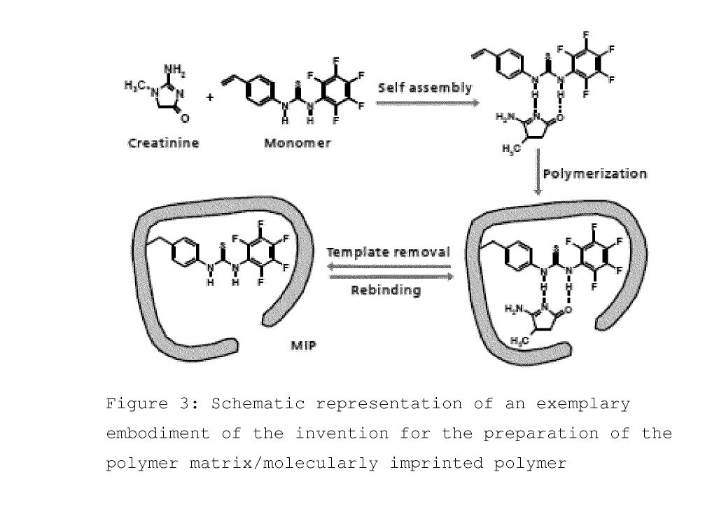

FIG. 4 shows a flow scheme of the indirect Jaffe reaction.

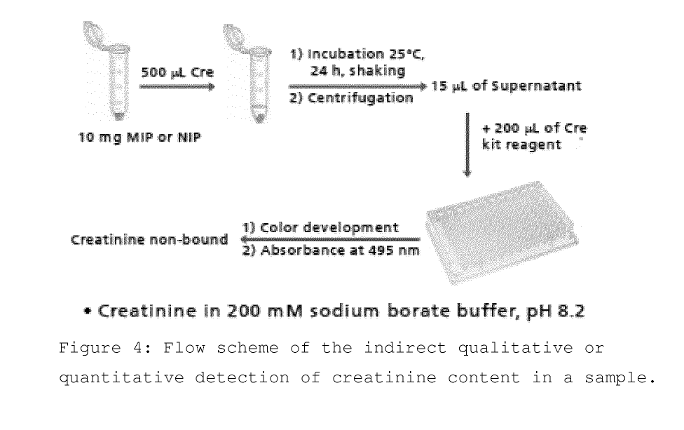

FIG. 5 shows the creatinine detection by Jaffe reaction from creatinine standard solution.

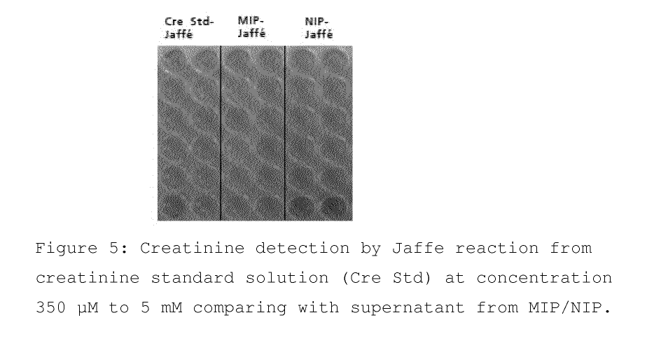

FIG. 6 illustrates the selectivity of the polymer matrix/MIP

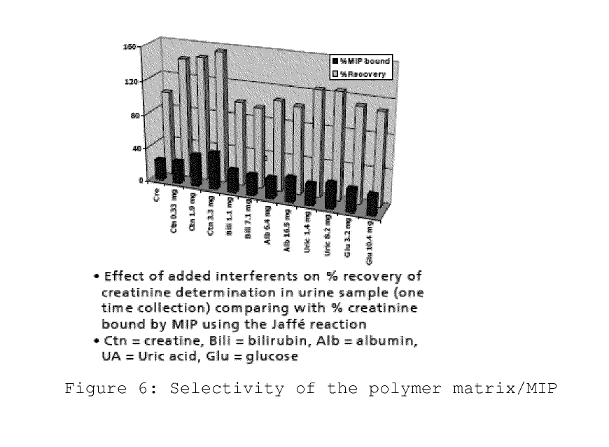

### EXAMPLES

### Example 1

### Synthesis of Functional Monomer, 1-(4-vinylphenyl)-3-(pentafluorophenyl) thiourea, PTU

1-(4-vinylphenyl)-3-(pentafluorophenyl) thiourea is synthesized and used as the novel receptor for neutral nitro derivatives in the previous article (Athikomrattanakul, U., Promptmas, C., Katterle, M., Tetrahedron Lett., 50: 359-362 (2009)).

### Example 2

### Synthesis of Molecularly Imprinted Polymers According to the Noncovalent Approach

In the present invention provided the molecularly imprinted polymers, which is prepared by the noncovalent imprinting approach developed by Mosbach in document U.S. Pat. No. 5,110,833.

The imprinted polymer is prepared by mixing 0.05655 g of creatinine (0.5 mmol) and 0.1724 g of 1-(4-vinylphenyl)-3-(pentafluorophenyl)thiourea (PTU, 0.5 mmol) in 5 ml of anhydrous dimethylsulfoxide (DMSO) in 10 mL glass vial. The mixture is incubated for 2 hours at 25 degree C. to allow for self-assembly of the template-monomer complexes. Then 1.7870 g of pentaerythritol triacrylate (PETRA, 6 mmol) and 127 mg of 1-hydroxycyclohexyl phenyl ketone are added to the mixture. The mixture is purged with argon gas for 5 minutes before incubated at 4 degree C. for 2 hours. After preincubation, polymerization is initiated under ultraviolet light at 366 nm for 6 hours exposure so as to form a monolith.

The non-imprinted polymer is prepared by 0.1725 g of PTU (0.5 mmol) in 5 ml of anhydrous dimethylsulfoxide in 10 mL glass vial. The solution is incubated for 2 hours at 25 degree C. before adding 1.7890 g of pentaerythritol triacrylate (PETRA, 6 mmol) and 129 mg of 1-hydroxycyclohexyl phenyl ketone to the solution. The mixture is purged with argon gas for 5 minutes and then incubated at 4 degree C. for 2 hours. After the preincubation, polymerization is initiated under ultraviolet light at 366 nm for 6 hours exposure so as to form a monolith.

The imprinted and non-imprinted monoliths are ground in a ultra centrifuge mill (Retsch, ZM200) and then sieved. The particles size is between 25 and 75 micron. The polymers are washed with hot methanol in a Soxhlet apparatus overnight for template removal and dried for 4 hrs at 60 degree C. before using in the next step. The course of template removal is monitored with a UV-Vis spectrophotometer at a wavelength of 230 nm.

### Example 3

### Measurement of Creatinine Standard Solution by Jaffe Reaction

Creatinine standard solutions with varied concentrations from 0 to 5 mg/dl (0 to 442 μM) are prepared from creatinine standard stock solution (20 mg/dl from creatinine assay kit, Cayman, USA) in 200 mM sodium borate buffer, pH 8.2.

The creatinine concentration of each concentration is analysed by Jaffe reaction using creatinine assay kit reagent from Cayman, USA. The supernatant (15 μL) of nonbound compounds in each tube is taken and transfered in to 96-well microplate (F-bottom, Griener). 100 μL of the reaction buffer (from kit) and 100 μL of the color reagent (from kit) are added to the supernatant. The color of the supernatant is developed and then analysed the adsorbance at 495 nm using microplate reader (FLUOstar, Omega) at one and seven minutes. The average absorbance of each concentration of the analyte can be calculated by substracting the one minute absorbance reading from the seven minute absorbance reading. Then the adjust analyte absorbance is obtained by substracting the average absorbance from the absorbance of blank sample (0 mg/dL). The standard curve of creatinine is plotted and fitted with the linear regression. The y-intercept and slope are obtained from the linear equation and used for calculation of the creatinine concentration in the sample.

### Example 4

### Evaluation of the Creatinine Recognition by the Imprinted Polymers in Aqueous Solution

Creatinine standard solutions with varied concentrations from 0.010 to 5 mM are prepared in 200 mM sodium borate buffer, pH 8.2. The analyte (500 μL) of each concentration is added to 10 mg of the imprinted polymer in each eppendorf tube. The mixture is incubated at 25 degree C. under continuous shaking for 24 hrs to ensure that the equilibrium is reached before centrifuged at 10,000 rpm in 10 min. The supernatant (15 μL) of nonbound compounds in each tube is taken and transferred in to 96-well microplate (F-bottom, Griener). 100 μL of the reaction buffer (from kit) and 100 μL of the color reagent (from kit) are added to the supernatant. The color of the supernatant is developed and then analysed the adsorbance at 495 nm using microplate reader at one and seven minutes. The concentration of the nonbound analyte is determined using a calibration curve of creatinine standard solution. The amount of the analyte bound (B, μmol/g) to the imprinted polymer is evaluated by subtracting the concentration of the non-bound analyte from the initial concentration. Each assay is determined in triplicate.

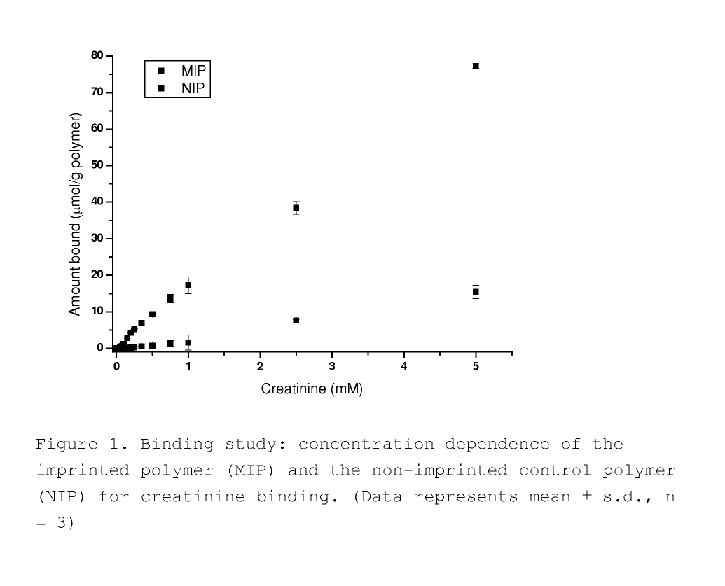

The result is illustrated by FIG. 1.

Under the analytical condition used, substantial recognition by the imprinted polymer is observed for creatinine at concentration above 50 μM (0.57 mg/dL).

The selective factor or imprinting factor value is determined for evaluation the recognition of creatinine on the matrices.

### Example 5

### Evaluation of Creatinine Recognition by the Imprinted Polymer in Real Sample (Urine)

A urine sample secreted by one of the authors is collected 2 hours before the test. Some of the urine sample spiked with various concentration of creatinine from 5.62, 10.66, 26.45, 53.04, and 80.75 mg/dL are prepared. The creatinine concentration in the urine sample with and without spiked creatinine was determined by Jaffe reaction before adding to the imprinted polymer.

The urine sample (500 μL) with and without spiked creatinine is added to 10 mg of the imprinted polymer in each eppendorf tube. The mixture is incubated at 25 degree C. under continuous shaking. The supenatant of each sample is collected for 4 and 24 hrs and then analysed with Jaffe reaction. After 50-folds dilution, 15 μL of the diluted urine sample is developed color with the kit reagent and then analysed the adsorbance at 495 nm using microplate reader. The concentration of the nonbound creatinine in urine is determined using a calibration curve of creatinine standard solution. The creatinine concentration bound to the imprinted polymer is evaluated by subtracting the concentration of the non-bound analyte from the initial concentration. Each assay is determined in triplicate.

The binding capacity of the imprinted polymer is dependent on the elevated concentration of creatinine.

### Example 6

### Recognition and Selectivity of the Imprinted Polymer to Creatinine in Real Sample (Urine)

Various interferences are added to urine samples with various concentrations, for example, creatine (4.2, 24, 42, 79 mM), glucose (45, 78, 146 mM), bilirubin (3, 10, 20, 30 mM), albumin (10.7, 17.5, 27.4, 38.3 mg/mL) and uric acid (13.9, 31.1, 84.3, 157. mM).

The creatinine concentration in the urine sample with various interferences is determined by Jaffe reaction before addition to the imprinted polymer. Afterwards, 500 μL of the urine sample with interference is added to 10 mg of the imprinted polymer in each Eppendorf tube. The mixture is incubated at 25 degree C. under continuous shaking. The supernatant of each sample is collected for 4 and 24 hrs and then analysed with Jaffe reaction. After 50-folds dilution, 15 μL of the diluted urine sample is developed color with the kit reagent and then analysed the adsorbance at 495 nm using microplate reader. The concentration of the nonbound creatinine in urine is determined using a calibration curve of creatinine standard solution. The creatinine concentration bound to the imprinted polymer is evaluated by subtracting the concentration of the non-bound analyte from the initial concentration. Each assay is determined in triplicate.

The binding capacity of the imprinted polymer for creatinine in urine sample is observed and it is noted that the imprinted polymer is more selective and specific for creatinine than the other interferences.

### Example 7

### Use of Material as a Stationary Phase for Sample Separation

The MIP according to the invention, which is more particularly selective to creatinine, makes it possible to use as the stationary phase for separation and/or isolation of the target analyte. This application is also based on the fact that the imprinted polymer has a better retention for the template molecules than others. Therefore, the invention relates to the use of MIP for packing in the solid phase extraction (SPE) cartridge and then used for sample preparation and separation, or to concentrate creatinine in any solution, such as aqueous, serum, plasma or urine. In addition to this particularly advantageous application, the MIP in this invention can also packed in HPLC column for direct measurement of creatinine by HPLC.

### Example 8

### Use of Material as a Recognition Matrix with Fluorescence Effect for the Creatinine

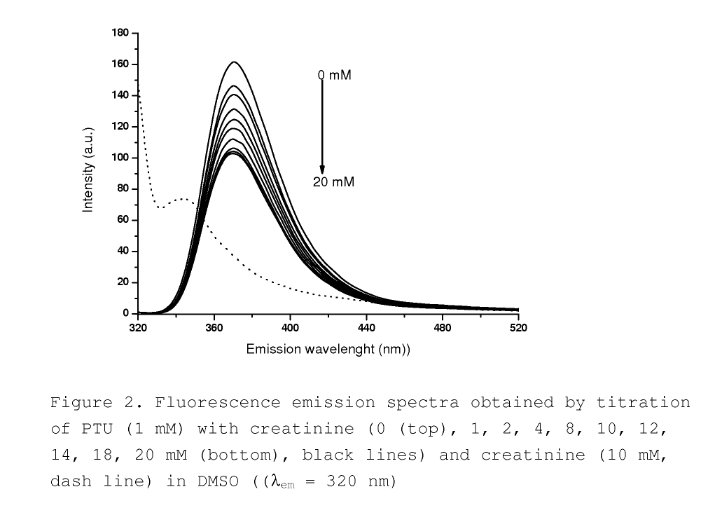

The functional group I′″ or functional monomer Ia′″, respectively, provided in this invention possesses intrinsic fluorescence properties that can be quenched by creatine solution as shown in FIG. 2. It is conceivable that the materials contain fluorescence units. Thus, the invention also relates to the use of the imprinted polymers as the recognition element, which can be prepared in other forms and coated on any surface of the transducer coatings for sensor application. Optical sensors prepared by the instant invention will provide quantitative measurement of creatinine levels via the changes in the fluorescence properties of the materials.

### Example 9

### Use of Material as a Recognition Matrix with Label-Free Measurements

This invention can be applied in label-free measurements for the target detection. For example, label-free measurements based on calorimetry have been introduced using a thermistor as the device for measuring the binding between MIPs and the specific target. For instance, a thermistor can be a flow-through device. A flow-through thermistor can comprise a packed bed reactor, for instance in the shape of a microcolumn. Thermoprobes can be present at the entrance and exit of the packed bed reactor for measuring the temperature.

In a flow-through thermistor the temperature difference between the entrance and exit can be continuously recorded and depends on the generated heat from enthalpy change due to adsorption and/or reaction inside the reactor, the total number of product molecules and the heat capacity of the system. This principle is described by Danielsson's group (Ramanathan, K., Danielsson, B., Biosens. Bioelectron., 16: 417-423 (2001)). For a given adsorption and/or reaction process between a analyte and a sorbent or catalyst in a given streaming media, e.g. water, buffered solution, organic solvent, at a fixed flow rate, the temperature difference depends only on the concentration of the analyte. In effect, the concentration of an analyte can be measured directly, in real time without the use of a label.

In the present example, a packed bed reactor was prepared by providing the polymer matrix comprising functional group I′″ of the present invention as a selective recognition matrix in a microcolumn with sample inlet and sample outlet of a flow calorimeter. The binding phenomenon between the creatinine and the imprinted polymer was detected from the thermometric response from the sample at the sample inlet and the sample outlet of the flow calorimeter. The thermometric response from the calorimeter allowed detection of the creatinine in micromolar concentrations.

Therefore, the combination of a flow calorimeter or thermoprobe with the polymer matrix of the present invention, e.g. provided in a packed bed column, can be used for label-free detection of creatinine.

Moreover, the label-free detection allows realisation of label-free sensing devices for the detection of creatinine. For example, a biosensor for label-free detection of creatinine can be realised.

1. Polymer matrix comprising at least one target binding site, wherein
the target binding site comprises a functional group represented by the general formula (I) 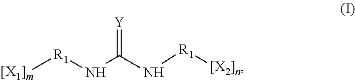
wherein
Y represents S, O or NH,
R1 and R2 are each independently selected from a group comprising aryl, heteroaryl, cycloalkyl, cycloalkenyl, alkyl, which can each be substituted and/or part of a condensed ring system,
X1 and X2 each independently represent a group comprising a chemical linkage to the polymer matrix, m and n independently represent 0, 1, 2 or 3 and m+n is 1, 2, 3, 4, 5 or 6,
the target binding site being suitable to bind to a target molecule represented by one of the following general formula (IIa) or (IIb): 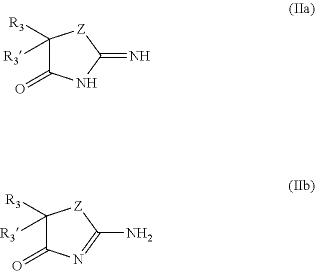
wherein Z is selected from CR4R4′, NR4″, O or S, R3, R3′, R4 and R4′ are each independently selected from a group comprising hydrogen, aryl, heteroaryl, cycloalkyl, cycloalkenyl, linear or branched alkyl, which can each be substituted and/or part of a condensed ring system,
R4″ is selected from a group comprising nitrogen protecting group, hydrogen, aryl, heteroaryl, cycloalkyl, cycloalkenyl, linear or branched alkyl, which can each be substituted and/or part of a condensed ring system.

2. Polymer matrix comprising at least one target binding site, wherein
the polymer matrix is obtainable by polymerization of at least a functional monomer of the general formula (Ia) 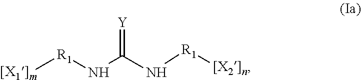
wherein
Y represents S, O or NH,
R1 and R2 are each independently selected from a group comprising aryl, heteroaryl, cycloalkyl, cycloalkenyl, alkyl, which can each be substituted and/or part of a condensed ring system,
X1′ and X2′ each independently represent a polymerizable group,
m and n independently represent 0, 1, 2 or 3 and m+n is 1, 2, 3, 4, 5 or 6,
the polymerization being performed in the presence of a target molecule represented by one of the following general formula (IIa) or (IIb): 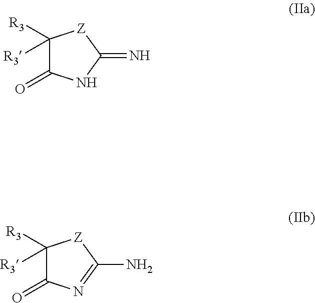
wherein Z is selected from CR4R4′, NR4″, O or S, R3, R3′, R4 and R4′ are each independently selected from a group comprising hydrogen, aryl, heteroaryl, cycloalkyl, cycloalkenyl, linear or branched alkyl, which can each be substituted and/or part of a condensed ring system,
R4″ is selected from a group comprising nitrogen protecting group, hydrogen, aryl, heteroaryl, cycloalkyl, cycloalkenyl, linear or branched alkyl, which can each be substituted and/or part of a condensed ring system,
wherein the target binding site of the polymer matrix is able to bind to the target molecule.

3. Polymer matrix of claim 2, wherein the polymerization is performed in the presence of at least one cross-linker and/or initiator.

4. The polymer matrix of claim 1, wherein the functional group of formula (I) is represented by the general formula (I′) 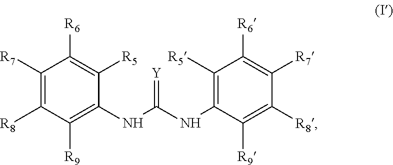
wherein R5, R6, R7, R8, R9, R5′, R6′, R7′, R8′ and R9′ each independently represent hydrogen, halogen, alkyl, halogenalkyl, NO2, CN, 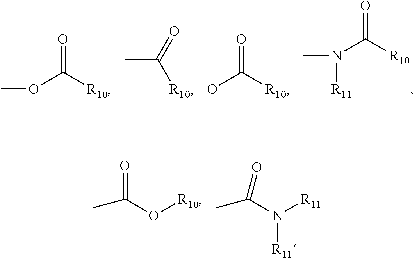
and R10, R11, R11′ being independently selected from a group comprising hydrogen, alkyl, aryl, halogenalkyl, alkylenaryl and nitrogen protecting group,
wherein at least two of R5, R6, R7, R8, R9 or at least two of R5′, R6′, R7′, R8′ and R9′can independently form part of a condensed ring system,
provided that at least one of R5, R6, R7, R8, R9 comprises X1 and/or at least one of R5′, R6′, R7′, R8′ and R9′ comprises X2.

5. The polymer matrix of claim 2, wherein the functional monomer of the formula (Ia) is represented by the general formula (Ia′) 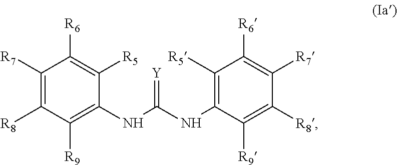
wherein R5, R6, R7, R8, R9, R5′, R6′, R7′, R8′ and R9′ each independently represent hydrogen, halogen, alkyl, halogenalkyl, NO2, CN, 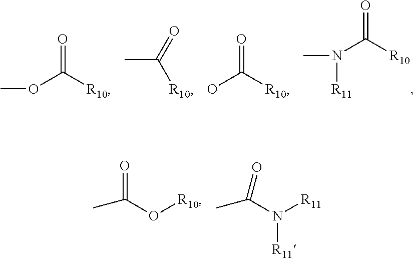
and R10, R11, R11′ being independently selected from a group comprising hydrogen, alkyl, aryl, halogenalkyl, alkylenaryl and nitrogen protecting group,
and/or wherein at least two of R5, R6, R7, R8, R9 or at least two of R5′, R6′, R7′, R8′ and R9′ independently form part of a condensed ring system,
provided that at least one of R5, R6, R7, R8, R9 comprises X1′ and/or at least one of R5′, R6′, R7′, R8′ and R9′ comprises X2′.

6. The polymer matrix of claim 1 or 4, wherein the functional group of formula (I′) is represented by the general formula (I″)

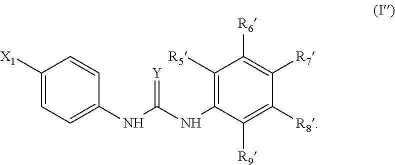

7. The polymer matrix of claim 2, 3 or 5, wherein the functional monomer of formula (Ia′) is represented by the general formula (Ia″)

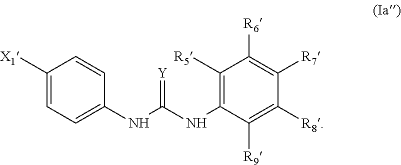

8. The polymer matrix of one of the claim 1, 4 or 6, wherein the functional group is selected from the group comprising:

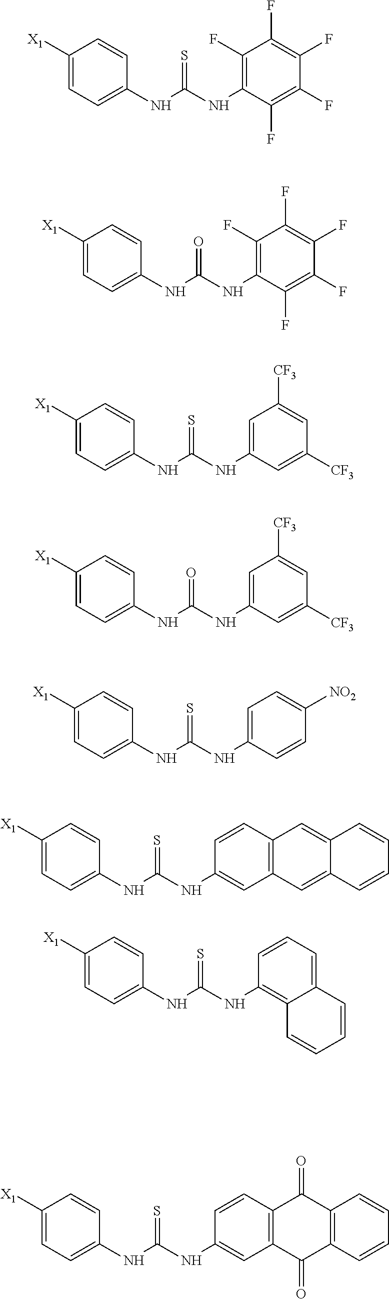

9. The polymer matrix of one of the claim 2, 3, 5 or 7, wherein the functional monomer is selected from the group comprising:

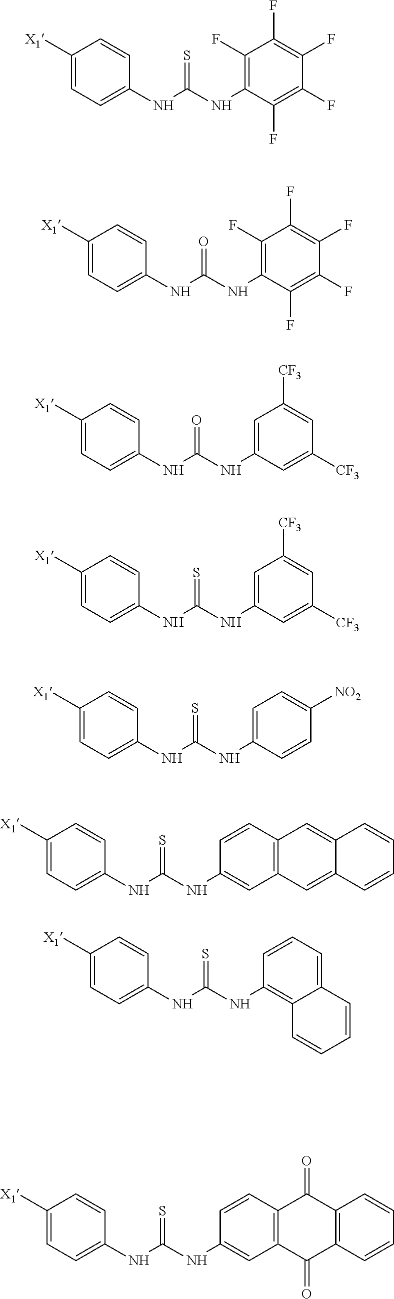

10. The polymer matrix of claim 1, 4, 6 or 8, wherein the functional group is represented by the formula (I′″)

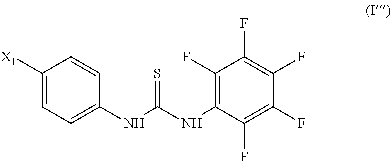

11. The polymer matrix of claim 2, 3, 5, 7 or 9, wherein the monomer is represented by the formula (Ia′″)

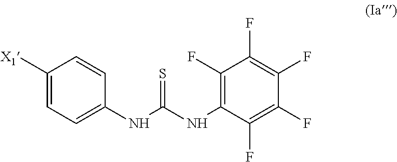

12. The polymer matrix of claim 1, wherein the functional group of formula (I) is represented by

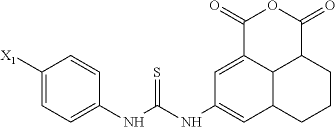

13. The polymer matrix of claim 2, wherein the functional monomer of the formula (Ia) is represented by

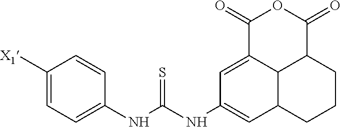

14. The polymer matrix of any of the preceding claims, wherein the target molecule is creatinine.

15. The polymer matrix of any of the preceding claims, wherein the polymer matrix is a co-polymer.

16. The polymer matrix of any of the claim 2, 3, 5, 7, 9, 11 or 13, wherein the polymerisable groups X1′ and X2′ are chosen from a group comprising unsaturated groups capable of reacting via a free-radical process, and chemical groups enabling a polycondensation, polyaddition or sol-gel reaction, in particular acryl derivatives, methacryl derivatives epoxides, isocyanates and allyl derivatives.

17. The polymer matrix of any of the claim 1, 4, 6, 8, 10 or 12, wherein the X1 and X2 are derived from a polymerization reaction of a group comprising unsaturated groups capable of reacting via a free-radical process, and chemical groups enabling a polycondensation, polyaddition or sol-gel reaction, in particular acryl derivatives, methacryl derivatives epoxides, isocyanates and allyl derivatives.

18. A method for the preparation of a polymer matrix as defined by any of the claims 1-17, comprising the method steps of:
a) providing the at least one monomer according to one of the claim 2, 5, 7, 9, 11 or 13 and at least one target molecule according to one of the claim 1, 2 or 12,
b) forming a pre-polymerization mixture by bringing said monomer and said target molecule into contact,
c) polymerizing said pre-polymerization mixture to obtain the polymer matrix.

19. The method of claim 18, wherein at least one cross-linker and/or initiator is added to the pre-polymerization mixture of method step b).

20. The method of claim 18 or 19, further comprising method step d) removing the target from the polymer matrix.

21. A method for the separation or extraction of creatinine from a mixture comprising additional organic and/or inorganic compounds, comprising the method steps of:
a1) providing the polymer matrix as defined by any of the claims 1-17,
b1) contacting the polymer matrix with the mixture and binding of creatinine with the polymer matrix,
c1) separating the polymer matrix with the bound creatinine from the mixture.

22. The method of claim 21, further comprising method step d1) releasing the bound creatinine from the polymer matrix.

23. The method of claim 21 or 22, wherein the mixture is a biological specimen.

24. The method of the previous claim, wherein the biological specimen is serum or urine.

25. A method for indirect qualitative or quantitative detection of creatinine content in a sample using the polymer matrix as defined by any of the claims 1-17, the method comprising the steps of:
a2) separating a sample to be analyzed into a first and a second separate portion,
b2) using a detection method for determining a first creatinine content result in the first portion of the sample in the absence of the polymer matrix,
c2) adding the polymer matrix to the second portion of the sample,
d2) using the same detection method for determining a second creatinine content result in the second portion of the sample,
e2) substracting the second creatinine content result from the first creatinine content result to obtain the creatinine content in the sample.

26. The method of the previous claim, wherein the detection method of method step b2) and d2) is selected from a group comprising spectrophotometric, colourimetric, fluorescence or electrochemical detection reactions.

27. The method of claim 25 or 26, wherein the detection method is a colourimetric detection reaction.

28. The method of one of the claims 25-27, wherein the detection method is based on the reaction of creatinine with alkaline picrate, in particular wherein the detection method is the Jaffe reaction.

29. An assay kit for performing the detection method of any of the claims 25-27 comprising
the polymer matrix as defined by any of the claims 1-17
means for detecting the target molecule with the method of one of the claims 25-28, wherein an assay kit is used for the detection method.

30. A method for detection of creatinine content in a sample using the polymer matrix as defined by any of the claims 1-17 without use of any further labelling group with the polymer matrix, the creatinine and the sample, comprising the method steps of:
a3) providing the polymer matrix,
b3) contacting the polymer matrix with the sample to be analyzed,
c3) measuring the interaction of creatinine with the polymer matrix.

31. The method of the previous claim, wherein method step c3) comprises calorimetric measuring of heat generated by an enthalpy change due to adsorption of creatinine to the polymer matrix and/or reaction of creatinine with the polymer matrix.

32. The method of the previous claim, wherein method step a3) comprises providing the polymer matrix in a packed bed having a sample inlet and a sample outlet, method step b3) comprises flowing the sample through the packed bed via the sample inlet and sample outlet, thereby contacting the polymer matrix with the sample, and method step c3) comprises measuring the sample temperature from the sample inlet and the sample temperature from the sample outlet and deriving the creatinine content in the sample from the difference between the sample temperature at the sample inlet and the sample temperature at the sample outlet.

33. The method of claim 30, wherein method step c3) comprises measuring a spectral characteristic of the polymer matrix in contact with the sample.

34. The method of the previous claim, wherein measuring the spectral characteristic comprises measurement of the fluorescence intensity of the polymer matrix in contact with the sample.

35. The method of the previous claim, wherein the fluorescence intensity is measured with an excitation wavelength of 300-340 nm, preferably 310-330 nm, most preferred 315-325 nm, and an emission wavelength of 350-400 nm, preferably 360-390 nm, most preferred 365-375 nm.

36. A sensing device configured for detection of creatinine content in a sample, comprising:
the polymer matrix as defined by any of the claims 1-17
means for measuring the interaction of creatinine with the polymer matrix,
wherein measuring the interaction of creatinine with the polymer matrix is without use of any further labelling group with the polymer matrix, the creatinine and the sample.

37. The sensing device of the previous claim, wherein the means for measuring the interaction of creatinine with the polymer matrix comprises a device configured for calorimetric measuring of heat generated by an enthalpy change due to adsorption of creatinine to the polymer matrix and/or reaction of creatinine with the polymer matrix.

38. The sensing device of claim 36, wherein the means for measuring the interaction of creatinine with the polymer matrix comprises a device configured for measuring of the fluorescence intensity of the polymer matrix in contact with the sample.
